(19) United States
(12) Patent Application Publication
(10) Pub. No.: US 2006/0020258 A1
(43) Pub. Date: Jan. 26, 2006

(54) Surgical apparatus with a manually actuatable assembly and a method of operating same

(75) Inventors: Paul Strauss, Grapevine, TX (US); Wade Phil Farrow, Bryan, TX (US); Rob Ellins, Lakewood, OH (US)

Correspondence Address: HAYNES AND BOONE, LLP, 901 MAIN STREET, SUITE 3100, DALLAS, TX, 75202, US

(73) Assignee: Medtronic, Inc., Minneapolis, MN (US)

(21) Appl. No.: 10/977,649

(22) Filed: Oct. 29, 2004

Related U.S. Application Data
(60) Provisional application No. 60/589,399, filed on Jul. 20, 2004.

(51) Int. Cl. A61B 17/00

(52) U.S. Cl. 606/1

(57) ABSTRACT

A surgical tool assembly and a method of operating same according to which a surgical tool is connected to a handpiece having an electrically operated motor; and a portable source of electrical energy is provided in a pedal assembly for controlling the tool. The source is electrically connected to the motor; and the pedal can be engaged for controlling the flow of the electrical energy from the source to the motor.

### CROSS-REFERENCE

This application claims priority of provisional application Ser. No. 60/589,399, filed Jul. 20, 2004.

### FIELD OF THE INVENTION

This invention relates to a surgical tool assembly utilizing a manually-controlled switch assembly, and a method of operating same.

### BACKGROUND

Many surgical tool assemblies include a motorized handpiece to which a cutting accessory, such as a drill bit, bur, saw blade, reamer, or the like, is attached, for removing or separating sections of body tissue. The handpiece is often connected to a separate switch for activating the handpiece, and to a separate, stand-alone, console. The console is connected to a source of AC power and contains electronics that converts the AC line voltage to a voltage suitable for driving and controlling the operation of the handpiece.

However, the console is bulky, somewhat complicated to set up, and is not very mobile. Also, at least two electric cables must be connected between the console and the switch, and between the console and the handpiece, respectively.

Other arrangements of the above type do not use a console but rather mount the above electronics in the handpiece. However, the handpiece must be autoclaved before each use to sterilize it, which can have a deleterious effect on the electronics.

All patents and patent applications listed in Table 1 are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the teachings of the present invention.

TABLE 1

| Patent/Publication No./ | Patented/Published | |
|---|---|---|
| Serial No. | Date/Filed | Inventor |
| 2004/0155132 A1 | August 12, 2004 | McPherson et al. |
| 10/699,474 | October 31, 2003 | Farrow et al. |

### SUMMARY

The above problems are solved by an embodiment of the present invention, in which the console is eliminated and a manually-actuatable switch assembly is provided that contains a portable source of electrical energy, and is adapted to control the operation of the motor. This assembly is relatively small, utilizes only one cable, is relatively easy to set up and is more mobile for office-based procedures, as well as procedures in remote settings. Also, the assembly does not have to be sterilized.

Various embodiments of the invention discussed below may possess one or more of the above features and advantages, or provide one or more solutions to the above problems.

### BRIEF DESCRIPTION OF THE DRAWINGS

FIG. 1 is a partial elevational/partial diagrammatic view of an embodiment of the present invention.

FIG. 2 is a plan view of a component of the embodiment of FIG. 1.

FIG. 3 is a diagrammatic view of the electrical connections between the components of FIG. 1.

### DETAILED DESCRIPTION OF THE PREFERRED EMBODIMENTS

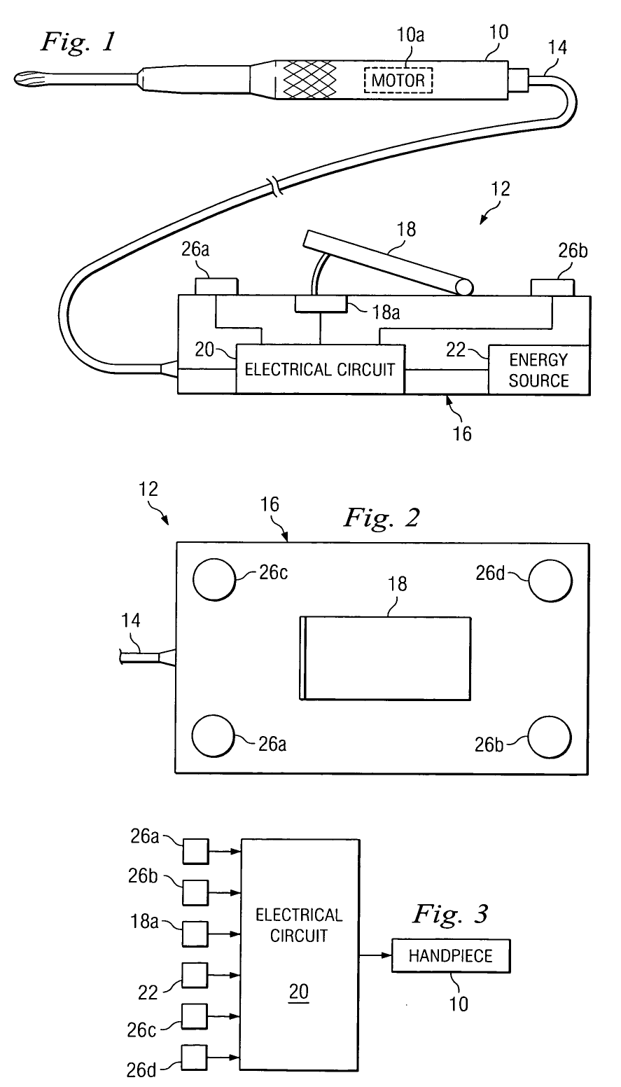

Referring to FIGS. 1 and 2 of the drawings, the reference 10 refers, in general, to a handpiece in the form of an electrically powered tool for use in surgical procedures, such as the removal or separation of body tissue. The handpiece 10 is driven by an internal electrical motor 10a, and is adapted to receive a cutting accessory (not shown), such as a drill bit, a bur, a saw blade, a reamer, or the like, that can be removably connected to the output shaft of the motor 10a in a conventional manner. When the motor 10a is activated in a manner to be described, the output shaft, and therefore the cutting accessory, are rotated, reciprocated, and/or oscillated at a predetermined speed to enable the surgical procedure to be performed.

A switch and control assembly 12 is provided for activating the motor 10a and controlling the operation of the handpiece 10 in a manner to be described. An electrical cable assembly 14 is electrically and mechanically connected between the assembly 12 and the handpiece 10, and includes a plurality of insulated electrical conductors that function in a manner to be described.

As shown in FIGS. 1 and 2, the assembly 12 includes a housing, or base member, 16 located a distance from the handpiece. A pedal 18 is pivotally mounted to the base member 16 in a manner so that at least a portion of the pedal is elevated relative to the base member. The pedal 18 is shown in its normal, non-pressed position but can be manually pressed downwardly towards the base member 16, by the foot or hand of the operator. After being pushed downwardly and released, the pedal 18 will return to its raised, or inactive, position by a spring, or other biasing device (not shown).

A switch 18a is mounted in the base member 16, is electrically connected in an electrical circuit 20 provided in the base member, and responds to movement of the pedal 18 for changing an electrical characteristic of the circuit in a manner to be described.

A portable source of electrical energy, such as DC power, 22 is also contained in the base member 16 and is connected in the electrical circuit 20. Preferably, the source 22 is a reusable or rechargeable energy source which could be in the form of a rechargeable battery, such as a lithium ion or nickel cadmium battery. To this end, it is understood that the base member 16 can include a jack, or terminal, (not shown) for receiving a cable leading from a electrical charger (not shown) which can be a dedicated charger or a charger from another surgical device, for charging the source 22.

One or more of the electrical conductors in the cable assembly 14 are connected between the electrical circuit 20 and the motor 10a of the handpiece 10 to establish a path for the flow of electrical energy from the source 22 to the motor. The design is such that when the pedal 18 is in its normal, raised position shown in FIG. 1, the switch 18a, and therefore the electrical circuit 20, are open; and when the pedal 18 is manually pressed downwardly towards the base member 16 by the operator, the switch 18a and the electrical circuit 20 are closed. In this closed condition of the electrical circuit, electrical energy from the source 22 flows through the electrical circuit 20 and to the motor 10a to power, or energize the motor. It is understood that the switch 18a and the electrical circuitry 20 associated with the pedal 18 can allow the speed of the motor 10a to be varied within its selected range by varying the degree of pressing of the pedal, in a conventional manner.

A series of four push-button/switch assemblies 26a, 26b, 26c, and 26d are provided on the base member 16 and are connected to the electrical circuit 20 to control various operational modes of the motor 10a, including changes in direction of movement, speed, speed ranges, and the like. The push button of each assembly 26a-26d can be manually pressed downwardly towards the base member 16 by the foot of the operator, and, after being released, will return to its raised, or inactive, position by a spring, or other biasing device (not shown). Each assembly 26a-26d includes an electrical switch (not shown) which is normally open but closes when the associated push button is pressed downwardly to control one of the above operating modes of the handpiece 10. Since the assemblies 26a-26d are conventional they will not be described in further detail. The electrical circuit 20 contains electronics that respond to the closing of the switch assemblies 26a, 26b, 26c, and 26d and send a corresponding signal to the motor 10a, via the electrical conductors of the cable assembly 14, to control one of the above-mentioned operational modes.

In operation, the surgeon attaches a cutting tool to the handpiece 10 and manually presses, or forces, the pedal 18 downwardly with his or her foot or hand. This closes the switch 18a and thus allows the electrical energy from the source 22 to flow to the motor 10a, via the electrical circuit 20 and the cable assembly 14, to activate the motor. Each push button assembly 26a-26d can be selectively activated by pressing it downwardly in the above manner to control one of the above-mentioned operational modes of the handpiece 10.

Thus, the arrangement of the present invention provides for activation and control of the operation of the handpiece 10 without the need for a console. Also, the above embodiment is relatively easy to set up, is mobile, and requires only a single cable assembly. Further, the electrical energy source and the control electronics are located in the housing that contains the pedal, which does not have to be autoclaved.

### Variations

It is understood that several variations may be made in the foregoing without departing from the scope of the invention including the following.
(1) The pedal 18 and its associated switch 18a are not limited to the particular types described above but rather can be one of many other conventional designs such as multi-pedal, snap action, slow action, potentiometer, air switch, etc., and can function to include maintained contact, single stage, two stage, three stage, mechanical interlock, and variable stage. Also the switch 18a can include two or more momentary type contacts which are sequentially activated as the pedal 18 is pressed.
(2) The movement of the tool that is attached to the handpiece 10 is not limited to a rotation, but rather can be an oscillation or reciprocation, and the like, or can use a water jet, or the like.
(3) The switch 18a and the switches associated with the assemblies 26a-26d can be connected in electrical circuitry that is normally closed and opened when the pedal and the associated push button assembly is pressed down in the above-described manner.
(4) The electrical circuit 20 may be contained in the handpiece 10 rather than in the base member 16.
(5) The push-button/switch assemblies 26a-26d can vary in number, depending on the number of operating modes desired, and can even be eliminated.
(6) Although the energy source 22 would typically be a reusable or rechargeable energy source, such as a battery, is could also be in the form of a disposable energy source.
(7) When replacement, reusable or rechargeable, or primary/disposable energy sources, such as the types of batteries discussed above, are not available, it would be possible to provide electrical power via an AC to DC power converter module, which would receive 120 volt AC power and convert it to the appropriate voltage for the surgical device. A cabled plug or jack from the converter would plug into a receiving plug on the base member 16.
(8) An embedded display could be incorporated into the base member 16 for status conditions (battery management, motor condition), surgeon preferred device settings, and/or current speed.
(9) Rather than have one pedal (18), the assembly 12 can include multiple pedals for providing separate functions of the type discussed above, in which case the number of buttons could be reduced.
(10) The pedal 18 and the push button assemblies 26a-26d can be either foot or hand operated.
(11) The energy source can be a hybrid layer capacitor, rather than a battery.

The preceding specific embodiments are illustrative of the practice of the invention. It is to be understood, therefore, that other expedients known to those skilled in the art or disclosed herein, may be employed without departing from the invention or the scope of the appended claims. For example, the present invention is not limited to any particular power driven surgical tool and further includes within its scope methods of making and using the system described hereinabove.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

What is claimed is:

1. A surgical apparatus comprising:
a handpiece for receiving a surgical tool and having an electrically operated motor;
a housing located a distance from the handpiece;
a portable source of electrical energy disposed in the housing; and
a manually actuated electrical switch supported on the housing and electrically connected to the source; and
an electrical cable electrically connecting the switch to the housing so that manual actuation of the switch controls the flow of electrical energy from the battery to the motor to control the operation of the motor.

2. The assembly of claim 1 wherein the switch includes a pedal mounted to the housing for movement relative to the housing when manually engaged, to actuate the switch to control the flow of the electrical energy.

3. The assembly of claim 2 wherein the switch, the source, and the motor are connected in an electrical circuit which is normally opened and closed by the switch in response to manual actuation of the pedal.

4. The assembly of claim 3 wherein the pedal, the switch, and the electrical circuit permit the speed of the motor to be varied in response to the degree of pressing of the pedal.

5. The assembly of claim 3 further comprising at least one additional, manually actuatable switch connected in the circuit for controlling a function of the motor.

6. The assembly of claim 5 wherein at least one of the additional switches controls the direction of movement or the speed of the tool.

7. The method of claim 1 wherein the portable source of electrical energy is a battery and the electrical energy is in the form of DC power.

8. The assembly of claim 1 wherein the switch is normally open to prevent the flow of the electrical energy, and is adapted to close in response to movement of the pedal to permit the flow of the electrical energy.

9. The assembly of claim 8 wherein the switch returns to its open position after being manually actuated.

10. A surgical method comprising:
connecting a surgical tool to a handpiece having an electrically operated motor;
providing a portable source of electrical energy in a housing located a distance from the handpiece;
electrically connecting the source to the motor;
manually moving a pedal on the housing; and
providing a switch that responds to movement of the pedal for controlling the flow of the electrical energy from the source to the motor in response to the movement of the pedal.

11. The method of claim 10 further comprising connecting the switch, the source, and the motor in an electrical circuit which is normally opened and closed by the pedal.

12. The method of claim 11 wherein the pedal, the switch, and the electrical circuit permit the speed of the motor to be varied in response to the degree of pressing of the pedal.

13. The method of claim 11 further comprising connecting at least one additional, manually actuatable, switch in the electrical circuit for controlling a function of the motor.

14. The method of claim 13 further comprising controlling the direction of movement of the tool by one of the additional switches.

15. The method of claim 10 wherein the switch is normally opened to prevent the flow of the electrical energy, and is adapted to close in response to movement of the pedal to permit the flow of the electrical energy.

16. The method of claim 15 wherein the switch returns to its open position after being manually actuated.
